(19) United States
(12) Patent Application Publication
(10) Pub. No.: US 2024/0342431 A1
(43) Pub. Date: Oct. 17, 2024

(54) WEARABLE DUAL-FUNCTION EEG INTEGRATED BONE CONDUCTION AUDIO DEVICE

(71) Applicants: Ryan Farzam Ahmed, Kew Gardens, NY (US); Authoy Das, Jamaica, NY (US); Shahriar Siam Huda, Jamaica, NY (US); Pari Patel, Hagerstown, MD (US); Dang Nguyen, Wellington, FL (US); Genevive Chang, New York City, NY (US); Mohammad Rahim, Jamaica, NY (US)

(72) Inventors: Ryan Farzam Ahmed, Kew Gardens, NY (US); Authoy Das, Jamaica, NY (US); Shahriar Siam Huda, Jamaica, NY (US); Pari Patel, Hagerstown, MD (US); Dang Nguyen, Wellington, FL (US); Genevive Chang, New York City, NY (US); Mohammad Rahim, Jamaica, NY (US)

(73) Assignee: Niura Corporation, Plainview, NY (US)

(21) Appl. No.: 18/633,526

(22) Filed: Apr. 12, 2024

Related U.S. Application Data
(60) Provisional application No. 63/495,630, filed on Apr. 12, 2023.

(51) Int. Cl. A61M 21/02; A61M 21/00; G06F 3/01; G06F 3/16; H04R 1/10

(52) U.S. Cl. CPC A61M 21/02; G06F 3/015; G06F 3/165; H04R 1/1008; H04R 1/1041; H04R 1/1083; A61M 2021/0027; A61M 2230/10; H04R 2460/01; H04R 2460/13

(57) ABSTRACT

The present invention relates to an auditory device that combines bone-conducting earbuds with integrated electroencephalography (EEG) sensors. The device utilizes bone conduction technology to transmit sound through the skull to the inner ear, allowing for clear audio perception without obstructing the ear canal. Incorporated EEG sensors are configured with spring-loaded contacts that maintain consistent skin contact to monitor brain wave activity effectively. This dual-functionality enables users to listen to audio and simultaneously track cognitive states, making the device particularly useful for applications in cognitive load monitoring and adaptive audio feedback based on the user's mental state. This innovation extends the functionality of conventional earbuds with bone-conduction mechanisms by integrating EEG technology to enhance biosensing, health monitoring, and tailored cognitive-processing feedback.

### RELATED U.S. APPLICATION DATA: CONVERSION OF PROVISIONAL (UTILITY)

Patent Application No. 63/495,630 Filed on Apr. 12, 2023

### PRIOR PUBLICATION DATA

US/2023/63/495,630

### FIELD OF CLASSIFICATION SEARCH

CPC . . . A61B 5/0476; A61B 5/24; A61B 5/6803; G06F 19/00; G16H 40/67; H04R 1/02; H04R 1/10; H04R 25/00;

REFERENCES CITED:

| | |
|---|---|
| 4,967,038 A1 October 1990 Gevens | 128/644 |
| 6,067,464 A1 May 2000 Musha | 600/383 |
| 6,154,669 A1 November 2000 Hunter | 600/383 |
| 20,170,078,780 A1 December 2017 Qian | H04R I/O |
| 008,792,975 B2 July 2014 Kato | 600/544 |

### FIELD

The invention pertains to the biopsychological detection of neural oscillations omitted during continuous monitoring of brain activity, particularly through a novel system and method for integrating electroencephalography (EEG) technology into bone conduction apparatuses. The present invention utilizes iterations of spring-loaded metal electrodes and accompanying software models for dual-purpose electric brain signal detection and audio delivery. Quantitative metrics received from the present disclosure have various biomedical, audio, fitness, entertainment, and productivity-enhancing applications. Thus, the accompanying invention aims to intersect wireless wearable technology and non-invasive EEG technology.

### BACKGROUND

Description of the Problem: Electroencephalography (EEG) provides people with biometric information regarding seizures, aneurysms, sleep problems, and more. In addition to providing individuals with information about their brain health, EEG data can inform people about various mental markers. EEGs detect waves of various frequencies ranging from delta, theta, alpha, beta, and gamma, that indicate aspects of cognitive function such as cognitive efficacy, attention levels, information processing capabilities, memory, and wakefulness. While comprehensive, EEG data is inaccessible to everyday people because only licensed technicians under accredited medical institutions can administer EEG readings. Given current protocol, individuals must also place their bodies under physical constraints such as dehydration, hunger, and sleep deprivation to acquire EEG data. EEG technicians will apply various creams and gels to ensure accuracy and conductivity from the specified brain lobe under the electrodes, and place a cumbersome cap on the user's head above the conductive gel. Under these strenuous conditions, the readings may not reflect each individual's most natural state. Due to the inaccessible nature of EEG technology and skewed information that may be collected during this test, individuals cannot monitor their daily cognitive health. This leaves room for abnormalities to infiltrate an individual's life and present chronic or acute problems. Bone-conduction headphones would be a suitable form factor for EEG technology, as their headband and around-the-ear format fit snugly against the user's head and ears to ensure accurate data collection while prioritizing user comfort. These “bonephones” transmit vibrations through the user's skull rather than the typical sound waves transmitted through their ear canal. In-ear earbuds capitalize on air conduction to transmit sound to the cochlea through the ear canal. The external ear (pinnae) collects sound vibrations, channels them to the middle ear through the ear drum, and stimulates the cochlea's fluids to generate the hearing sensation. Conversely, bone-conduction headphones decode electrical input from the terminal and translate that into vibrations that stimulate the cochlea directly, with frequencies ranging from 0.25 and 8 kHz, subverting the path through the outer and middle ear. Bone conduction technology was originally implemented to aid those affected by hearing loss, especially those with acute middle ear damage. However, bone conduction technology has since been used in many general consumer devices with audio playback capabilities. As a result, bone conduction headphones can increase accessibility to biometric data from the brain, especially EEG data.

Description of the Related Art and Limitations: While the biometrics field has developed novel electrocardiogram and authentication system applications, EEG (electroencephalogram) technology has not progressed similarly since its conception during the early 20th century. Traditional EEG methods an array of electrodes placed on the scalp to detect brain signals and are primarily used in controlled environments. Their size and sensitivity to external factors such as perspiration or movement can affect the accuracy of readings. These devices generally require specialized interpretation, which limits their accessibility

Recent efforts to innovate EEG technology emphasize compactness and portability, but compromise on accuracy and comprehensiveness. These new models use fewer electrodes and are localized in specific brain areas to improve specific aspects of individuals' functionality. However, scalp-based electrodes face interference from hair, sweat, and natural oils, interfering with signal accuracy. To address this, newer technologies are exploring in-ear EEG systems that move away from traditional scalp placements to potentially increase comfort and portability.

In ear-based biometric wearables, traditional designs attach metallic or conductive components to the external parts of earbuds. These designs do not use the silicone of the earbud tips as electrodes, nor do they integrate electrode arrays within the ear with significant surface area. Some older models place electrodes on external hooks, which changes the appearance and function of typical consumer earbuds. The current technology aims to overcome these challenges by revising EEG design to improve utility and user experience.

Objective of the Invention: The proposed invention aims to integrate EEG (electroencephalography) detection into bone conduction technology to capture brainwaves from the frontal and temporal regions of the brain. The device is capable of audio transmission through bone vibrations while simultaneously capturing EEG signals. The invention includes electrodes integrated into the bone conduction transducer, ensuring accurate data collection, and a processor to analyze EEG activity and dynamically adjust audio output. The device provides real-time cognitive state feedback and environmental noise cancellation for an optimized user experience in various contexts. The device resolves accessibility issues in a compact form factor that is easy for individuals to use and retain.

Brief Summary of the Invention: The invention is a wearable auditory device with EEG-integration that transmits sound through bone conduction. Multiple electrodes, including ground and conducting electrodes, will be integrated into the bone conduction transducer. These electrodes are spring-loaded to maintain contact with the user's skin and are coated with conductive material to ensure accurate data collection. Additionally, these electrodes will be coupled with a processor to analyze EEG activity and adjust audio output accordingly. The processor implements machine learning algorithms to interpret the EEG data. This wearable auditory system comprises a biofeedback mechanism that provides real-time feedback to individuals about their cognitive state and environmental noise cancellation capabilities that adjust based on the user's environment to optimize experience.

### Technical Terminology and Concepts:

Electroencephalogram (EEG): A screening and diagnostic method employed to measure and record electrical activity generated by the brain. This is done using electrodes placed on the scalp, which capture fluctuations in voltage resulting from ionic current flows within the neurons of the brain.

Electrodes: Conductive devices or mediums designed to detect electrical signals. In the context of the present invention, these are integrated into earbuds to facilitate non-invasive capture of EEG data.

EEG-Integrated Earbuds: A novel integration of EEG technology into earbuds. This involves the incorporation of electrodes within the earbuds' structure, enabling the continuous capture of EEG data while performing the primary function of audio delivery.

Alpha, Beta, Theta, Delta, and Gamma Waves: Different classifications of brainwaves based on their frequencies. Each type is associated with specific mental states or functions and can provide insights into brain health and activity.

EEG Amplifier: A component responsible for enhancing the electrical signals captured by EEG sensors. It ensures that the signals are of sufficient strength and clarity for subsequent processing and analysis.

Amplitude: In the context of EEG data, amplitude refers to the magnitude of the electrical signal or brainwave at any given point in time.

Frequency: Represents the number of oscillations or cycles of a wave per unit of time. In EEG, it helps in categorizing brain electrical signals into types like Alpha, Beta, etc.

Shape and Duration: These are graphical representations and lengths of time, respectively, for specific brain electrical signals or patterns. Variations in these parameters can offer insights into potential neurological anomalies or states of consciousness.

### SUMMARY

Electroencephalography (EEG) caps and machines are limited in their scope of usage, as they require individuals to place their bodies under strenuous physical conditions to adhere to medical protocol. As data is collected in clinical settings, this data may not reflect each individual's most natural state. Later iterations of EEG devices prioritize portability while sacrificing accuracy, comprehensiveness, and functionality.

The invention comprises an innovative bone-conductive headphone system that integrates electroencephalogram (EEG) technology with audio output. This device allows for real-time monitoring of brain activity while delivering sound through bone conduction, offering insights into cognitive states alongside high-quality audio experiences. Its design conforms to the user's skull and ears, protecting wiring from external damage while prioritizing the user's comfort and experience. This device contains bone conduction transducers that direct sound transmission through a sound-conducting pad, through the skull, and to the cochlea, bypassing the ear canal and allowing users to remain aware of their environment. EEG electrodes remain in continuous contact with the user's skin, and are coated with a conductive material to gather brain activity data with minimal signal interference for processing. They are specifically placed to monitor frontal and temporal lobes, offering insights into cognitive processes like attention and memory. The processor is engineered to evaluate the user's cognitive state, which in turn informs the adjustment of the audio output. The device can modify volume, equalization, or audio content based on the user's cognitive state as determined through EEG data analysis. Moreover, the device contains a biofeedback mechanism that provides the user with instantaneous feedback about their cognitive state.

The integration of EEG technology into bone conduction headphones serves to increase users' access to EEG data, which provides them with insights into their brain health and general cognition. With our processor's In operation, FIG. 1 portrays the integration of EEG and audio functions—the sound is wirelessly delivered to the processing unit and amplified while EEG sensors monitor brain activity. This simultaneous action captures brainwaves and provides real-time feedback to customize audio output based on cognitive states and surroundings. The processors provide users with feedback that may benefit them in biomedical, audio, fitness, entertainment, and productivity-enhancing applications.

### BRIEF DESCRIPTION OF DRAWINGS

In the following portion relating to the detailed description, the embodiments of the present disclosure will be explained more in detail with reference to the example figures of the proposed invention shown in the drawings, which:

FIG. 1 illustrates a potential application of the present disclosure, featuring a lateral-view perspective of the EEG-integrated bone-conducting device with electrodes on the skin-contact pad.

FIG. 2 illustrates a potential application of the present disclosure, featuring a lateral-view perspective of the EEG-integrated bone-conducting device with electrodes on the ear loop and skin-contact pad.

FIG. 3 presents a lateral-view perspective of the proposed invention on an individual with highlighted electrodes, transducers, and apparatus.

FIG. 4 depicts an exploded view of the present disclosure, highlighting the necessary processors, electrodes, transducers, BLE components, and other materials presented.

FIG. 5 displays a schematic representation of the presented invention once user input has been finalized and the proposed device is in optimal conditions for operation.

### DETAILED DESCRIPTION OF DRAWINGS

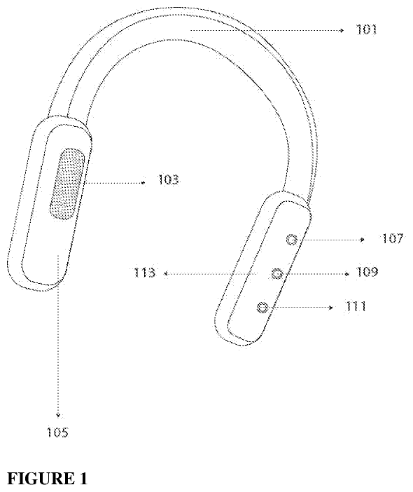

FIG. 1 illustrates the inventive bone-conductive headphone that incorporates EEG technology with auditory functionality. This dual-purpose device offers non-invasive brain activity monitoring while providing audio output through bone conduction, ensuring that users can assess cognitive states in real-time and experience high-fidelity sound transmission.

The device comprises a headband (101), which serves as the primary support structure. It not only secures the device firmly on the user's head but also discreetly houses the essential wiring for the device's electronic components. Attached to the headband is the bone-conducting transducer housing (103), positioned externally near the ear. This housing contains the transducers responsible for converting audio signals into mechanical vibrations. These vibrations are then transferred to the sound-conducting pad (105), which directly interfaces with the skin and facilitates the conduction of sound vibrations through the skull to the cochlea, bypassing the external auditory canal.

Embedded within the device are several spring-loaded metal electrodes: electrode 1 (107), electrode 2 (109), and electrode 3 (111). These electrodes are crucial for the EEG functionality of the device, capturing electrical brain activity from specific regions of the brain. The spring-loaded mechanism ensures consistent contact with the skin, adapting to any movement and maintaining signal quality. The electrodes are housed within the electrode housing apparatus (113), which is situated adjacent to the sound-conducting pad (105). This configuration allows for the concurrent collection of neural data and delivery of auditory information, providing a comprehensive monitoring and listening experience.

The headband 101 is the foundational structure that secures the device on the user's head. 101 contours to the shape of the skull, ensuring stability when used as intended, and conceals essential wiring for the device's electronic components to safeguard against external damage and preserve the headphone's integrity. Its ergonomic design minimizes pressure points, providing a comfortable fit for extended periods of wear.

In direct contact with the user's skin, the sound-conducting pad, 103, is a critical component for the propagation of sound vibrations from the transducer 405 to the skull.

The bone conducting transducer housing, 105, vibrates against the skull to direct sound waves to the cochlea, bypassing the ear canal and eardrum and directly stimulating the cochlea. The position of 105 allows for simultaneous voice communication functionality and environmental awareness, allowing the device to serve as a comprehensive tool for personal audio and interactive engagement with the surroundings.

The electrodes 107, 109, 111 maintain constant contact with the user's skin to serve as touchpoints for EEG data acquisition. Coated with biocompatible, conductive material, 107, 109, 111 minimize signal impedance and capture the brain's electrical activity emanating from the brain. 107, 109, 111 are positioned to monitor the frontal and temporal lobes, areas associated with cognitive functions such as attention, memory, and emotion.

In operation, FIG. 1 demonstrates the seamless integration of EEG monitoring with audio functionality. As 113 detects sound waves, it wirelessly transmits the audio input to the processing unit. Simultaneously, 103, stimulated by electrical signals from the connected device, vibrates against the skull. These vibrations are then transmitted through 105 to the cochlea, enabling sound perception while bypassing the ear canal.

During this process, 107, 109, 111 engage in continuous EEG monitoring. 107, 109, 111 detect the electric brain activity from the brain's regions closest to the contact points. The spring-loaded mechanism ensures that the electrodes maintain optimal contact, accounting for any movement by the user, which is critical for accurate EEG data capture.

The collected EEG data is then amplified and processed by an integrated circuit within 101, employing machine learning algorithms to decipher brainwave patterns. The system can thus provide real-time feedback on cognitive states and adapt the audio output, including noise cancellation and audio modulation, to enhance the user's experience based on the EEG readings and environmental conditions.

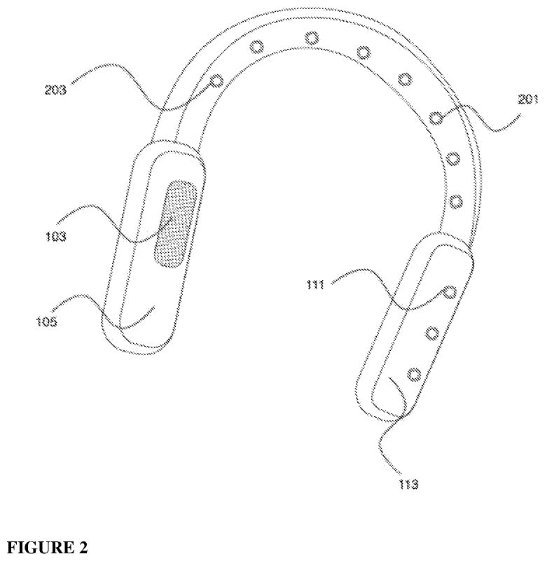

FIG. 2 displays the top down view of the bone-conductive headphone apparatus designed to combine EEG monitoring with auditory transmission capabilities. This depiction focuses on the placement of various components critical to the device's dual-functionality, emphasizing their arrangement around the ear-loop and the interface with the user's head.

FIG. 2 provides a side view of the presented invention that melds auditory and neuro-monitoring technologies. Key to its functionality are the spring-loaded metal electrodes, with Electrode 1 (201) and Electrode 2 (203) positioned on the ear loop, serving as ground electrodes for detecting electric brain activity. These electrodes are strategically placed to ensure the capture of neural signals from the brain's regions adjacent to the ears.

Central to the device's auditory capability is the bone-conducting transducer housing (103), situated externally near the ear. Within this housing are transducers that convert audio signals into mechanical vibrations. These vibrations pass through the sound-conducting pad (105), which remains in contact with the skin, allowing for the direct conduction of sound to the user's cochlea through the skull.

The device's neuro-monitoring capacity is further enhanced by additional ground electrodes situated on the sound-conducting pad—Electrode 1 (107), Electrode 2 (109), and Electrode 3 (111)—which together capture a comprehensive profile of the user's brain activity. Each electrode is designed to maintain a constant connection with the skin, despite any movement by the user, ensuring the accuracy of the EEG data collected.

Additionally, the device may include an external microphone grill (113), an optional component that captures ambient sounds for situational awareness or to facilitate voice communication during calls. This feature ensures that the user remains connected to their environment while engaging with the present invention.

Electrode 1, 201, and Electrode 2, 203, are situated on the ear loop and detect brain activity. Its spring-loaded design ensures precise contact with the user's skin for capturing electrical brain activity accurately from the cerebral regions adjacent to the electrode placement. 201 and 203's strategic placement is determined by neurophysiological mapping to optimize the reception of electrical signals associated with brain function.

105 acts as the medium through which the vibrations from 405 are imparted to the user's skull.

107, 109, 111 are mounted on 113 and are part of the EEG monitoring array. Each spring-loaded metal electrode is tailored to maintain optimal skin contact, adjusting to individual anatomical differences and movements to collect consistent brainwave data. These electrodes are essential in detecting and measuring the various frequency bands of brain activity, from delta to gamma waves, which inform on cognitive states such as focus, relaxation, and alertness.

In FIG. 2, each component is positioned to fulfill its function without hindering the performance of others. 201, 203, 107, 109, and 111 are distributed to balance the EEG data collection with the ergonomics of wearing the device. 103 is designed for minimal contact with the skin to avoid damping the sound vibrations while still ensuring that the sound-conducting pad (105) can effectively transmit them. This design approach ensures that the device is compatible for extended wear and that the EEG and auditory functions can operate simultaneously without interference.

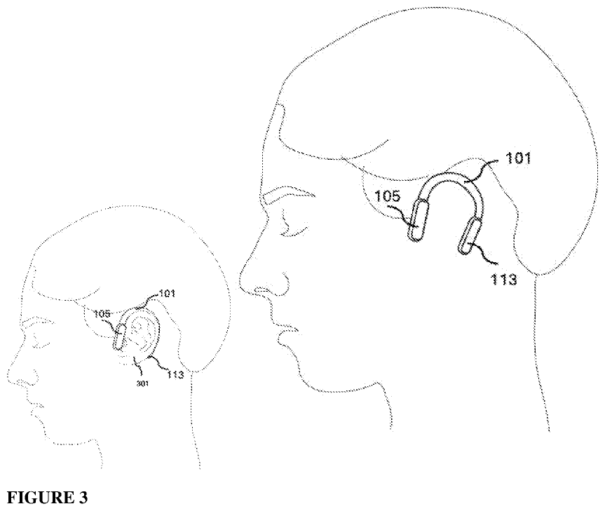

FIG. 3 illustrates a lateral and rear-side perspective of a user wearing a bone conduction headphone system integrated with EEG monitoring technology. The system includes a headband 101 designed to be worn over the top of the user's head, providing structural support for the attached components and ensuring stability and comfort during use. The headband is engineered to contain the necessary wiring and circuitry required for the device's operation, which may include connections to the bone conduction transducers 405, EEG electrodes, and any onboard processors or communication modules. The headband is designed to conform to various head shapes and sizes, ensuring a secure fit while accommodating the electronic wiring integral to the device's functionality.

Just in front of the ear 301, the bone conduction transducers 405, would be housed within the component marked as 105, which in this figure represents the interface pad that contacts the skin. These transducers are responsible for translating the electronic audio signals into mechanical vibrations. These vibrations bypass the traditional air conduction route through the ear canal, instead, directing the acoustic energy through the skull to stimulate the cochlea directly, facilitating an auditory experience without obstructing the ear canal.

Below the transducer housing and near the ear 301, the electrode housing apparatus 113 is visualized. This feature secures in place multiple spring-loaded electrodes (not visible in this view) integral to the EEG functionality of the device. The spring-loaded design of these electrodes is critical for maintaining a consistent and dependable connection with the skin, essential for the precise capture of EEG data used to monitor brain activity and cognitive states.

The right view in FIG. 3 provides a rear perspective, further elucidating the positioning of the headband 101 as it extends behind the ear. This angle also affirms the placement of the sound-conducting pad 105 and the electrode housing apparatus 113, emphasizing their roles in both the conduction of sound and the acquisition of EEG data.

Combined, these views detail the ergonomic integration of the device's audio and EEG monitoring systems, illustrating the wearing of the device and the non-invasive nature of the technology. The design is indicative of the device's dual capabilities: providing high-fidelity audio through bone conduction and capturing comprehensive neural activity data through EEG sensors for real-time cognitive state assessment. This innovative approach demonstrates the device's utility in a range of applications, from personal health monitoring to enhancing user interaction in various environments.

The bone conduction transducers 405, positioned adjacent to the ears, are encapsulated within a specialized housing. These transducers are responsible for converting electrical audio signals into mechanical vibrations. These vibrations are conducted through the bones of the skull, bypassing the ear canal to stimulate the cochlea directly, thereby facilitating the perception of sound without the use of the ear canal.

In direct contact with the skin behind the ear, the sound-conducting pad 105 serves as a medium for transmitting the vibrations generated by the transducers 405 to the user's skull. The pad is designed for optimal vibrational transfer.

Below the transducer housing, the device features an electrode housing apparatus 113, which secures the spring-loaded electrodes. These electrodes are crucial for capturing EEG data, with the spring-loaded mechanism ensuring consistent skin contact. This contact is key to obtaining high-quality EEG signals, which are then analyzed to monitor the user's cognitive state.

The EEG monitoring function captures brain activity through these electrodes, which detect the electric signals produced by the brain. The electrodes' spring-loaded nature compensates for any variation in pressure or movement, maintaining signal quality. Data collected from these electrodes are then processed and analyzed to provide feedback on the user's cognitive state.

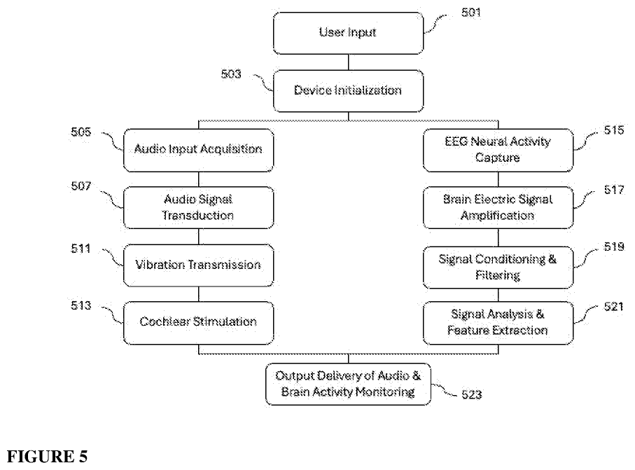

In addition to the numbered components, FIG. 5 suggests the presence of additional internal mechanisms and features, such as vibration dampeners to minimize noise interference and enhance audio clarity, as well as processors to manage audio signal processing, wireless connectivity, and user interface controls.

This device intersects the private audio experience with cognitive monitoring, offering potential applications across various contexts, including health monitoring, fitness, entertainment, and productivity enhancement. The invention's ergonomic design and non-invasive EEG integration make it a tool in wearable technology, providing users with access to real-time biometric and cognitive feedback.

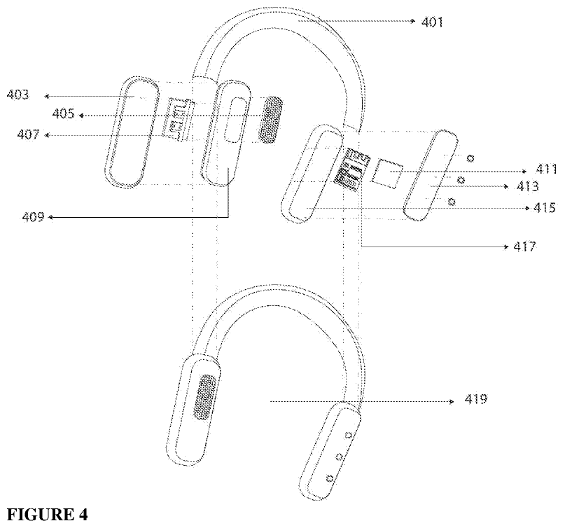

FIG. 4 provides a posterior view of the current invention. This representation reveals the integration and spatial arrangement of various components that constitute the device, each playing a pivotal role in its dual functionality of sound transmission and brainwave monitoring.

101 is the skeletal framework that unites all headphone elements into a single cohesive unit, ensuring that the headphones maintain their shape, and provides a secure fit.

Vibration dampeners 403 are integrated into the presented invention to mitigate unwanted vibrations and reduce noise. As bone conduction headphones play frequencies ranging from 0.25 and 8 kHz, 403's placement allows it to absorb excess energy from the transducers 405, which could otherwise result in auditory distortion or discomfort during prolonged usage.

At the core of the device's auditory capabilities are the bone-conduction transducers 405. 405 converts electrical audio signals into mechanical vibrations, which are transmitted directly through the user's skull to the inner ear, bypassing traditional air conduction through the ear canal. 405 consists of a vibrating element within a compact enclosure. When an electric audio signal is applied to 405, it induces mechanical vibrations in the vibrating element through electromagnetic or piezoelectric effects. These vibrations are then transferred to the adjacent bone tissues upon contact, bypassing the external ear canal entirely. As the mechanical vibrations propagate through the bones of the skull, they stimulate the cochlea, the auditory organ responsible for hearing.

The transducer mounting bracket 407 is a key structural component that secures 405 in its precise operational location. This ensures optimal alignment for effective sound conduction and mitigates any potential shift that could affect performance.

Serving as the exoskeleton for the internal workings, 105 protects 405 and internal circuitry from external environmental factors. Moreover, the 409 design is acoustically considered to enhance the transmission of vibrations and safeguard the device's sound quality against external noise interference.

The battery 411 is chosen based on the desired balance between runtime, size constraints, and the power demands of the integrated EEG technology.

The soft padding 413 houses 107, 109, 111 in optimal placement, ensuring they maintain consistent contact with the user's skin for continuous and accurate EEG monitoring, critical for tracking cognitive states.

The housing for the control circuit board 415 compartment is designed to encase the control circuit board 417, providing mechanical protection and electromagnetic shielding. It supports 417 securely while allowing for heat dissipation and easy access for maintenance or upgrades.

417 is a high-density assembly populated with microprocessors, wireless communication modules (including BLE capabilities), and battery management systems. 417 is responsible for interpreting EEG signals, processing audio, and managing power distribution and connectivity. 417 is designed to accommodate a multitude of components, including electrodes, amplifiers, filters, and microcontrollers, within a compact and densely populated layout. Microcontrollers and signal processing units are integral components of 417, responsible for digitizing, analyzing, and interpreting the EEG signals. 417 implements complex algorithms for signal processing, feature extraction, and data analysis, enabling real-time monitoring and feedback.

Additionally, high-density assembly PCBs may incorporate wireless communication modules, such as Bluetooth or Wi-Fi, for data transmission to external devices such as smartphones or computers. This facilitates remote monitoring, data logging, and further analysis of EEG signals. 105 makes direct contact with the user's skin, transferring vibrations from the transducers 405 through the skull and to the cochlea.

FIG. 5 illustrates the operational flow of a pair of bone-conducting earbuds with integrated EEG monitoring capabilities. Initially, user input (501) is required to activate and set preferences for the device, initiating device initialization (503), where the device powers up and performs self-check routines to ensure all systems are operational.

For audio processing, the device captures external audio input (505), such as music or phone calls, which is then transformed by audio signal transduction (507) into a compatible digital format for processing. The bone-conducting transducers inside take these digital signals and convert them into mechanical vibrations (509). These vibrations are transmitted directly to the cochlea, bypassing the outer and middle ear, thereby stimulating the inner ear and allowing the user to perceive sound through vibration transmission (511).

In parallel, the earbuds' EEG functionality is engaged. EEG neural activity capture (513) involves the collection of electrical activity from the user's brain via sensors. These brain signals are typically faint, requiring amplification (515) to ensure they are discernible for further processing. Signal conditioning and filtering (517) are then applied to the amplified brain signals to remove extraneous noise and artifacts, ensuring that the data represents neural activity.

After the signals have been cleaned, signal analysis and feature execution (519) take place. This sophisticated process involves interpreting the EEG data to extract meaningful information related to the user's cognitive state. Based on the analysis, specific features can be executed, such as adjusting the audio output to enhance the listening experience or providing feedback on the user's cognitive state, like alertness or relaxation.

Finally, the present disclosure outputs both the audio and the results of the brain activity monitoring (521). The audio is delivered through the bone conduction pathway, while the EEG monitoring may influence the audio experience or serve other applications based on the user's brain activity, effectively combining an auditory device with neurotechnology for a multifaceted user experience.

1. A wearable auditory device, comprising:
a. a bone conduction transducer configured to transmit sound to a user's inner ear through bone vibration;
b. at least one type of electrode integrated with the bone conduction transducer, wherein the electrode is configured to detect electrical activity in the user's brain; and
c. a processor communicatively coupled to at least one EEG sensor, wherein the processor is configured to analyze the electrical activity detected by the EEG sensor and adjust the audio output of the bone conduction transducer based on the analyzed electrical activity.

2. The wearable auditory device of claim 1, wherein:
a. the adjustment of audio output includes altering volume, equalization, or audio content in response to the user's cognitive state as determined by the EEG sensor.

3. The wearable auditory device of claim 1 or 2, further comprising:
a. a communication interface configured to transmit data related to the electrical activity detected by the EEG sensor to an external device.

4. A method for adjusting audio output in a wearable auditory device, the method comprising:
a. detecting electrical activity in a user's brain using at least one EEG sensor integrated with a bone conduction transducer of the wearable auditory device;
b. analyzing the detected electrical activity to determine a cognitive state of the user.

5. The wearable device of claim 1 or 2, wherein the EEG electrodes are spring-loaded to maintain contact with the user's skin, compensating for motion or pressure variation to ensure consistent signal quality.

6. The wearable device of claim 1 or 2, wherein the electrodes are coated with conductive material that enhances the electrical conductivity between the electrode and the user's skin.

7. The wearable auditory device of claim 1, wherein the processor is further configured to use machine learning algorithms to interpret the electrical activity detected by the EEG sensor, thereby improving the accuracy of cognitive state determination over time.

8. The wearable auditory device of claim 1 or 2, further comprises environmental noise cancellation capabilities that adjust based on the user's current cognitive state or environmental noise levels to optimize experience without compromising the user's awareness of their surroundings.

9. The wearable device of claim 1 further comprises a biofeedback mechanism that provides real-time feedback to the user about their cognitive state, enabling the user to engage in self-regulation exercises or mindfulness practices.
